(19) United States
(12) Patent Application Publication
(10) Pub. No.: US 2024/0180837 A1
(43) Pub. Date: Jun. 6, 2024

(54) Method for Treating Inflammatory Skin Conditions and other Topical Conditions or Disorders

(71) Applicant: Marc Selner, North Hills, CA (US)

(72) Inventor: Marc Selner, North Hills, CA (US)

(21) Appl. No.: 18/531,539

(22) Filed: Dec. 6, 2023

Related U.S. Application Data
(60) Provisional application No. 63/475,701, filed on Dec. 6, 2022.

(51) Int. Cl. A61K 9/127; A61K 9/00; A61K 45/06; A61K 47/10

(52) U.S. Cl. CPC A61K 9/1277; A61K 9/0014; A61K 45/06; A61K 47/10

(57) ABSTRACT

The present invention is directed to transdermal drug delivery composition designed for the effective administration of a pharmacological agent having a formulation utilizing specially formulated ethosomes or transferosomes, enabling penetration of both the stratum corneum and dermis layers of the skin. The ethosomes, characterized by their non-polar and fat-soluble nature, incorporate isopropyl alcohol or ethanol as the alcohol component. Introducing lipid-based vesicles with remarkable proficiency in encapsulating medications and immunosuppressants. This unique composition significantly enhances the penetration of the stratum corneum layer. This ensures precise delivery to affected areas upon topical application. By transcending the limitations of conventional treatments, the disclosed method holds substantial promise for advancing therapeutic outcomes and elevating the overall patient experience.

### CROSS-REFERENCE TO RELATED APPLICATIONS

This application claims the benefit of Provisional Patent Application No. 63/475,701 filed Dec. 6, 2022, and entitled Water Soluble Cyclosporine A Dry Eye And Allergic Eye,” Provisional Patent Application No. 63/361,174 filed Dec. 3, 2021, and Provisional Patent Application No. 63/205,447 filed Dec. 14, 2020, the disclosures of which are hereby incorporated by reference.

### FIELD OF THE INVENTION

The invention relates to topical compositions that provide dermatological benefits, such as for the treatment of inflammation, itching, pain relief, and/or other conditions associated with skin disorders.

### BACKGROUND

Topical conditions or disorders encompass health issues that affect various external body surfaces, including the skin, mucous membranes, subcutaneous tissue, and underlying skin layers.

Inflammatory skin conditions, such as dermatitis and psoriasis, present persistent challenges for a significant global population. Dermatitis, a broad term encompassing skin inflammation, manifests in various subtypes, including atopic dermatitis (eczema), contact dermatitis, seborrheic dermatitis, perioral dermatitis, and neurodermatitis. These conditions can result in redness, inflammation, itching, and sometimes flaking or blistering, with causative factors including allergies, irritants, or genetic predisposition.

Psoriasis, a chronic autoimmune condition marked by the rapid overproduction of skin cells, inflicts profound devastation, especially when traditional topical treatments prove ineffective. Individuals often resort to biologic treatments, acknowledging potential drawbacks despite their demonstrated efficacy. The use of biologics introduces a nuanced landscape, as individuals may face adverse side effects ranging from mild reactions to more serious concerns like increased susceptibility to infections or potential impacts on vital organs. This delicate balance between seeking relief and navigating potential risks underscores the complexity of managing psoriasis when conventional approaches fall short.

Adding to the challenges is the significant burden of social anxiety stemming from visible skin disorders like psoriasis. The persistent and noticeable symptoms contribute to heightened self-consciousness, discomfort in social settings, and compromised self-esteem. Beyond the physical toll, individuals must grapple with the emotional impact of navigating a world where the visibility of psoriasis may trigger societal biases or misconceptions. This multifaceted challenge requires not only effective medical treatments but also a sensitive approach that considers both the physical and emotional well-being of individuals contending with the complexities of psoriasis. Living with psoriasis brings not only physical discomfort and psychological challenges but also a chronic nature that significantly impacts daily activities. The visible nature of the condition may lead to heightened self-consciousness and social stigma. Individuals also grapple with the chronicity of the disease, potential health complications, and the emotional toll of managing a long-term condition. Other examples of topical conditions also include other concerns such as, but not limited to, acne, clogged pores, abscesses, cysts, boils, or localized infections.

Topical therapies enable precise medication delivery to the affected area, minimizing dose and potential side effects associated with systemic treatments. However, not all topicals can effectively penetrate the skin, due to a lack of fat solubility, limiting their efficacy in treating certain skin conditions.

While alternative treatments abound, their efficacy is often constrained by limited effectiveness or adverse reactions. The conventional method, involving oral administration, results in an undesired systemic surge of drug concentration in the bloodstream. This not only compromises treatment efficacy but also raises concerns about potential toxicity. For instance, widely used oral immunosuppressants like cyclosporine A, methotrexate, and azathioprine, employed for severe cases of inflammatory skin conditions, may lead to toxic blood levels and organ damage, particularly in the liver and kidneys.

Thus, there is a need for improved treatment options that are safe, effective, and provide long-term relief for patients suffering from inflammatory skin conditions. Such an advancement in skin care technology has the potential to improve the quality of life for millions of people worldwide.

### SUMMARY OF INVENTION

The present invention introduces a groundbreaking composition and method for the topical treatment of dermatological conditions, presenting a substantial leap forward in the field of dermatology. This innovative approach holds the potential to alleviate the suffering of millions worldwide affected by various topical disorders. Through a meticulous combination of unique ingredients and a carefully orchestrated production process, the invention not only offers a secure and efficient alternative to traditional treatments but also stands poised to emerge as a pivotal instrument in the ongoing battle against dermatitis.

The present invention represents a significant advancement in the treatment of inflammatory skin conditions, that addresses the limitations of current treatment options by providing a method for effective medications to penetrate the skin and treat the target area, thereby avoiding the toxicity caused by oral, intravenous, or subcutaneous administration.

Described herein are unique compositions featuring lipid-based vesicles adept at encapsulating medications and immunosuppressants. This innovative formulation facilitates the penetration of the stratum corneum layer, ensuring targeted delivery to affected areas upon topical application. By overcoming the limitations of conventional treatments, this method provides a promising avenue for enhanced therapeutic outcomes and improved patient experiences.

The present invention provides a novel method for treating topical or superficial conditions, such as dermatitis or migraine pain as well as others, with a skin applied composition. More specifically the skin applied composition comprises an active drug composition carried in a nanovesicle, preferably ethosome or transferosome structure which is dispersed in a compatible delivery vehicle (including but not limited to cream, ointment, gel, methyl/ethyl cellulose dissolved in water) which is easily absorbed when applied to skin. Also disclosed are methods of making such compositions. It is understood where ethosome is used, transferosome may be substituted where appropriate.

By incorporating lipid-based vesicles in this composition, the delivery of medications or immunosuppressants through the user's skin is optimized. This method ensures effective penetration of the outermost skin layer, the stratum corneum, facilitating medication reach to the affected area when applied topically. This targeted delivery enhances the overall impact of the treatment.

The utilization of lipid-based vesicles such as ethosomes offers several advantages in this composition. They improve the bioavailability of drugs, shield them from degradation, and enhance drug delivery to the intended site. Additionally, these vesicles are biodegradable and biocompatible, ensuring their safety for use in the human body.

Furthermore, ethosomes play a crucial role in enhancing drug permeability through the stratum corneum barrier. This makes them valuable for administering drugs with poor skin permeation, low oral bioavailability, susceptibility to first-pass metabolism, and the need to target skin infections at their source.

Formulating the ethosomes requires a careful and systematic approach, presenting a technical challenge that demands precision in both ingredient selection and procedural execution. The intricacies involve striking a delicate balance among various components and addressing the potential hurdle of phase separation.

### DETAILED DESCRIPTION

The invention is directed to a composition utilizing nanovesicles, preferably ethosomes or transferosomes for drug delivery to better reach targeted areas. The drug dissolved in ethanol is encapsulated within the vesicle which is formed with phospholipids dissolved in isopropyl or another suitable alcohol exhibiting an amphipathic nature and formed by shocking the combined solutions with hot water, at a suitable temperature around 120° F.-150° F. (48.89° C.-65.56° C.), typically around 140° F. (60° C.) or any temperature suitable to have the solution form ethosomes. When using phospholipids like soy lecithin, this precise process leads to the formation of ethosomes, a distinct type of nanovesicle with an outer layer predominantly composed of phospholipids. Further additives to this solution can be coupled with a surfactant to make a transferosome (preferable for improved penetration), propylene glycol. The outcome is a notable increase in the efficacy of penetration through the stratum corneum, ensuring targeted delivery to specific areas. Furthermore, these therapies can be user-friendly, reduce the risk of accidental overdose, and can be administered by the patient at home, presenting a convenient treatment option for many individuals.

In the context of dermatological formulations, dermatological formulations, especially those designed to penetrate the stratum corneum, it is crucial to understand the intricate composition and properties of this protective barrier. The stratum corneum, composed mainly of dead skin cells and lipids, acts as a robust shield, safeguarding the body from the external environment.

Keratin, a vital component of the stratum corneum, plays a pivotal role in maintaining skin integrity. Its structure, characterized by folded disulfide bonds, contributes to both resilience and impermeability. Corneocytes, the cells responsible for stratum corneum production, arrange keratin and fat in alternating layers, forming fatty bilayers. This intricate arrangement creates a water-repelling barrier, essential for physiological homeostasis by preventing water ingress into the body.

To enhance the penetration of therapeutic agents through the stratum corneum, understanding the complexities of the fatty bilayers is critical. Opting for substances with fat-solubility becomes imperative, as they can act as channels through the hydrophobic layers, facilitating the effective delivery of pharmaceutical formulations into the skin. The present invention addresses challenges when the therapeutic drug lacks fat solubility, providing a mechanism for successful penetration through the stratum corneum. Novel formulations aim to overcome barriers, offering effective solutions for various dermatological conditions.

Additionally, the size, solubility, and charge of molecules play a crucial role in their ability to penetrate the skin. Large molecules or proteins may struggle to pass through tight skin junctions, while water-soluble drugs face challenges in penetrating the lipid layer. Charged molecules could interact with the skin's charged surface, leading to repulsion. Moreover, the formulation of the topical product influences its ability to penetrate the skin.

One method involves using small spherical ethosomes, encapsulating a variety of drugs and therapeutic agents, and penetrating the skin more effectively than other lipid-based vesicles due to their unique composition. Preferred embodiments include phospholipids and ethanol, making them more flexible and deformable. High ethanol content increases the fluidity of the skin's lipid bilayers, allowing ethosomes to pass through more easily.

Ensuring stability when ethosomes encounter skin water is crucial to prevent them from falling apart and coalescing. A hot water shock enhances shell locking, addressing the lack of shell locking or stabilizing. Zeta potential, representing ideal charges on the ethosome membrane, significantly improves penetration, with higher values contributing to stabilization. Formulating transferosomes with cationic surfactants further enhances the zeta potential. Polyglycols increase the penetration rate, delivering substances to deeper skin structures. Ethosomes outperform large, rigid liposomes in penetration.

To enhance entrapment efficiency, increase the amounts of phospholipid and alcohol. Ethosomes, involving phospholipid encapsulation with substances like soy lecithin, form a vesicle containing alcohols and water. The membrane should be soft, flexible, and capable of opening the fatty bilayers of the corneum, acting like a channel for penetration. The corneum inhibits absorption, but alternating fatty bilayers created by corneal cells allow penetration. Substances must be non-polar, small, flexible, and able to open the fatty channels for passage. Ethosomes facilitate the passage of non-fat-soluble substances through the fatty bilayer and deeper into the skin by enveloping the drug with fatty encapsulation. Unlike liposomes, ethosomes lack cholesterol, making them smaller, more flexible, and capable of passing through the corneum. Factors contributing to ethosome entrapment and stability include higher amounts of phospholipid and alcohol, stirring, mixing, ultrasound, and heat. Heat shocking with water enhances membrane stability, preventing coalescence and breakup when exposed to water.

Ethosomes typically have an average size of 1.112 μm, with a spherical shape bearing a smooth surface observed by transmission electron microscopy and surface electron microscopy. The maximum entrapment of ethosomes is usually around 90-94%. Formulating ethosomes at a small size (25 nm-1000 nm), typically less than 200 nm, preferably less than 100 nm in diameter, enhances their penetration into deeper skin layers, contributing to the improved skin penetration and delivery of therapeutic agents achievable with ethosomes. Creating stable ethosomes for beneficial topical medications can be a complex task, and the following methods simplify the process, demonstrating effective accomplishment for a variety of drugs and applications.

The foundational composition of the drug-filled vesicles initiates with the creation of a first and a second solution. This first solution combines warmed alcohol, preferably isopropyl although other bipolar alcohols such as a benzo alcohol, or possibly other liquids or other substances having the ability to interact with both water and nonpolar substances, with a phospholipid, preferably soy lecithin. In a preferred embodiment, the alcohol undergoes heating, enhancing solubility to approximately 20° F. (about 11.11° C.) or more above room temperature. In preferred embodiments, it is then blended with 1-4% by weight of phospholipid until fully combined, preferably homogeneous, yielding a solution with a distinctive yellowish hue. The specific proportions of alcohol to phospholipid can vary.

Optionally, depending on the end use, to this phospholipid-drug mixture can be added 5-10% propylene glycol, 1-15% glycerin, and 0.02-0.5% PHMB (surfactant) are added and stirred. Optionally, ultrasound may be employed to enhance solubilization. It is understood that specific combinations would be determined by the end use by those skilled in the art.

A second solution of an effective drug solution is prepared in a separate container by dissolving an effective amount of the drug in a suitable amount of ethanol so the drug fully dissolves and solubilizes in the ethanol, creating a solution. Typically, the range of powder (mg) to liquid alcohol (ml) is 0.1:1 to 2:5, with a preferred ratio of 1:5 powder to liquid alcohol, such as 0.1 mg of powder is 0.5 ml alcohol.

Following this, the phospholipid-alcohol solution is added to a suitable amount of the drug solution to combine into a mixture. It's crucial to maintain a suitable ratio as deviations may compromise the lipid vesicle infrastructure, while too much phospholipid-alcohol may result in skin irritation. Typically, the drug solution would comprise 0.01% to 0.5% concentration of the total solution which would include phospholipid-drug mixture depending on the concentrations of the phospholipid-alcohol solution which may have the optional additives or excipients listed above.

Subsequently, an effective amount of distilled hot water (heated to 120° F.-150° F. (48.8° C.-65.55° C.), typically around 140° F. (60° C.)) is added and stirred into the phospholipid-drug mixture, inducing a shock and forming an ethosome mixture containing the selected drug enveloped or encapsulated in lipid-based vesicles.

Further enhancements involve the addition of about 10 ml of glycol caprylic acid and glycol to the mixture after shocking to help the peptide penetrate the skin.

For end use, the mixture is placed into a suitable carrier for application onto the skin in a concentration that is effective in treating the underlying inflammatory skin condition. For example, a suitable carrier such as cellulose gel base is prepared for carrying the ethosome mixture for application to a user's skin. This involves heating at least a liter of water to approximately 120° F.(48.89° C.), adding about 15 to 60 grams of ethyl and methyl cellulose powder, and stirring until a thick gel with a syrupy consistency is formed. After the cellulose mixture cools to room temperature, 50 ml of the ethosome mixture is added slowly and mixed until uniform, resulting in the creation of a liter of gel/lotion.

These components collectively contribute to the innovative drug delivery system, emphasizing enhanced stability, skin-friendly attributes, and targeted efficacy in the treatment of dermatological conditions.

The disclosed embodiments encompass methodologies for fabricating ethosomes or other lipid-based vesicles, incorporating a biocide or other medicament, some of which are enumerated below. This formulation facilitates enhanced penetration owing to its surfactant and antimicrobial characteristics, thereby providing a targeted approach and an overall reduction in systemic absorption of the medication involved in the treatment.

In a specific embodiment, the generation of drug-filled vesicles involves combining separately created mixtures, resulting in a composition that, when shocked, transforms into an ethosome mixture. This resultant mixture can subsequently be incorporated into a gel or lotion for application to a user's skin.

Selection of the specific components requires careful consideration to ensure seamless compatibility with regard to polarity and solubility throughout the formulation process. The successful creation of the ethosome mixture is dependent on the harmonious interaction of these elements. In preferred embodiments, the formulation incorporates several components that synergistically enhance its efficacy:

Soy Lecithin (Phospholipid): Derived from soybeans, soy lecithin serves a dual role as a surfactant, improving stability and emulsion, and contributing to the structural integrity of phospholipid bilayers. Its biocompatibility facilitates enhanced drug delivery through improved penetration.

Propylene Glycol: This synthetic organic compound acts as a versatile vehicle, ensuring even distribution of the drug and contributing to the formulation's stability, consistency, and uniform application to the skin.

PHMB (Polyhexamethylene Biguanide): Serving as a preservative and antimicrobial agent, PHMB ensures the stability of the formulation over time by preventing microbial growth, crucial for maintaining integrity and preventing contamination in topical applications.

Within the context of topical formulations herein, the incorporation of substances like propylene glycol or PHMB (Polyhexamethylene Biguanide) play a dual role, not only as elements of the overall formulation as described above, but also enhancing the formulations with the capability of capillary action. The distinctive features of propylene glycol, a polyhydric alcohol, recognized for its humectant and solvent properties, contribute to the improved permeation of the formulation through the skin's layers. Similarly, PHMB, with its antimicrobial strength, not only ensures the formulation's stability but also assists in achieving capillary action. This effect, a valuable attribute in dermatological applications, underscores the deliberate and inventive nature of the disclosed formulation, enhancing its effectiveness through careful ingredient selection and their multifaceted contributions.

Glycerin, another polyhydric alcohol, recognized for its humectant properties, contributes moisturizing benefits, preventing dryness, and enhancing the sensory experience of the gel/lotion upon application. Widely used in skincare products, this ingredient acts as a humectant, retaining moisture

Glycol Caprylic Acid: Caprylic acid, a fatty acid, contributes to the stability of lipid-based vesicles, while glycol enhances the overall properties of the formulation, potentially influencing its texture and absorption characteristics.

Cellulose Gel (Ethyl and Methyl Cellulose): Ethyl and methyl cellulose, forming a cellulose gel, act as carriers for the ethosome mixture. They add viscosity and thickness, creating an effective medium for controlled drug release and skin adherence. This comprehensive combination ensures the stability, consistency, and therapeutic properties of the formulation, promoting skin-friendly attributes.

### Example 1—Dermatitis Treatment Ethosome Formulation

Making a treatment comprising cyclosporine A ointment capable of treating severe dermatitis, including psoriasis and eczema.

The foundational composition of the drug-filled vesicles commences with:
1. Prepare a first solution by dissolving 30 ml of soy lecithin with 1000 ml of warmed isopropyl alcohol warmed to about 86-104° F. (30-40° C.). The elevated temperature enhances the solubility of the soy lecithin in the alcohol, yielding an even mixture with a characteristic yellowish hue indicating that the soy lecithin is evenly dissolved in the alcohol.
2. Prepare a second solution in a separate container by dissolving an effective amount of 150 ml
3. Cyclosporin A, for example 1 gram, in ethanol (20 ml) using a pestle and mortar. The drug is fully dissolved and solubilized in the ethanol making a solution.
4. Add 150 ml of the phospholipid-alcohol mixture to the 20 ml drug mixture and combine.
5. To this now phospholipid-drug mixture, add 15 ml of propylene glycol, 30 ml of glycerin, and six drops of PHMB.
6. Stir the mixture until solubilized.
7. Add about 90 ml of hot water (heated to about 140° F. (60° C.) or more) and stir into the phospholipid-drug mixture, inducing a shock and forming an ethosome mixture containing the selected drug enveloped in lipid-based vesicles.
8. Add 10 ml of caprylic acid and glycol to the mixture.
9. Separately, prepare a cellulose gel mixture for carrying the ethosome mixture and application to a user's skin. This involves heating at least a liter of water to approximately 120° F.). (50° C.), adding about 15 grams of ethyl and methyl cellulose powder, and stirring until a thick gel with a syrupy consistency is formed. Once the cellulose mixture cools to room temperature, 50 ml of the ethosome mixture is added slowly and mixed until uniform, resulting in the creation of a liter of gel/lotion.

In an alternative embodiment 30-60 grams of cellulose can be used.

An alternative formulation of a preferred embodiment uses Alcohol 5-25%, Soy lecithin 1-4%, Distilled water, 10-20%, heated to shock from 122-158° F. (50-70° C.), Propylene glycol 5-25%, Balancing ethyl and methyl cellulose gel to make a liter.

### Psoriasis & Dermatitis

Cyclosporin A is an immunosuppressive medication. It is used to prevent the body's immune system from rejecting transplanted organs, such as a kidney, liver, or heart. Cyclosporine works by suppressing the immune response. In addition to its use in organ transplantation, cyclosporine has found applications in treating certain autoimmune conditions. It is sometimes prescribed orally for the management of autoimmune disorders like rheumatoid arthritis and psoriasis, where an overactive immune system is involved.

However, there are inherent risks associated with the conventional oral administration of Cyclosporin, where blood levels can easily surpass the critical range of 250-400 ng/ml. The distinct application of the Cyclosporine A ethosome formulation in dermatology emerges as a revolutionary approach to addressing topical conditions. This paradigm shift provides a safer alternative, mitigating the potential for problematic and potentially dire systemic consequences.

This Cyclosporine A ethosome formulation surpasses cortisone, ensuring safe usage for over a year without the adverse effects of skin atrophy, hormonal changes, or organ rejection. It demonstrates superior efficacy, treating psoriasis within four months and providing eczema relief in just one week.

This formulation operates safely without substantially elevating blood levels, unlike oral administration. Typically, 30 ml per month results in a minimal increase, raising blood levels to 6 ng/ml for every 30 ml used-considerably lower than the level needed to prevent organ rejection, which is set at 300 ng/ml. This reduced systemic exposure contributes to the present formulation's superior safety profile, minimizing the risk of systemic problems often associated with higher blood levels.

Cyclosporine A in its regular form is not typically used for the skin due to the potential for systemic problems, particularly when blood levels range from 250-400 ng/ml. This highlights the unique and safe application of this formulation in dermatological conditions. This composition also facilitates spontaneous drainage, providing a comprehensive and effective solution for dermatological issues.

A formulation of a preferred embodiment using Cyclosporin A is as follows. Mix 1 gram of cyclosporin A powder mixed with 3-10 grams ethanol. Add heated mixture of isopropyl alcohol 86° F. (30° C.) and 1-3% soy lecithin mix. Add 180 ml. to the cyclosporin A/alcohol mixture. Then add 120-240 ml. of propylene glycol and mix and 30 ml glycol caprylic acid. Add 120-180 ml of hot water all at once to shock mixture. Separately add 15 grams each of methyl and ethyl cellulose to warm water and stir until it becomes a gel. Add enough of this mixture to make a liter and stir. This method can be abated to make other immune modulators topical gels that are peptides.

### Example 2—Hair Growth Stimulating Transferosome Formulation

A dermatological composition for enhanced drug delivery, comprising:
1. Prepare a first solution by dissolving 30 ml of soy lecithin with 1000 ml of warmed isopropyl alcohol warmed to about 30-40° C. (86° F.-104° F.) The elevated temperature enhances the solubility of the soy lecithin in the alcohol, yielding a mixture with a characteristic yellowish hue.
2. Prepare a second solution in a separate container by dissolving 3 grams of finasteride, in 15 ml ethanol. The drug must be fully dissolved and solubilized in the ethanol making a solution.
3. Add 15 ml drug solution to at least 360 ml of the phospholipid-alcohol mixture and combine.
4. To this now phospholipid-drug mixture, 90 ml of propylene glycol, 30 ml of glycerin, and six drops of PHMB are added and stirred.
5. Following this, about 90 ml of hot water (heated to 140° F. (60° C.) or more) is added and stirred into the phospholipid-drug mixture, inducing a shock and forming an ethosome mixture containing the selected drug enveloped in lipid-based vesicles.
6. Further enhancements involve the addition of about 10 ml of caprylic acid and glycol to the mixture.
7. Separately, a cellulose gel mixture can be prepared for carrying the transferosome mixture and application to a user's skin. This involves heating at least a liter of water to approximately 120° F. (48.8° C.), adding about 15 grams of ethyl and methyl cellulose powder, and stirring until a thick gel with a syrupy consistency is formed. Once the cellulose mixture cools to room temperature, 50 ml of the ethosome mixture is added slowly and mixed until uniform, resulting in the creation of a liter of gel/lotion.

Finasteride serves as a potent DHT (dihydrotestosterone) blocker, strategically combating hair loss and stimulating hair regeneration. DHT, a key contributor to hair loss in those predisposed to androgenetic alopecia, is effectively inhibited by Finasteride. This action helps maintain a healthy environment for hair follicles, slowing down the progression of hair loss and fostering the growth of new, robust strands. The innovative delivery system provides a targeted solution for individuals looking to preserve and revitalize their hair.

In an alternative embodiment, Minoxidil can be used as the active drug component in the above formulation. Minoxidil, a well-established vasoactive agent, acts as a vasodilator in this formulation, promoting blood flow and circulation in the scalp. This enhanced blood supply is believed to nourish hair follicles, potentially fostering hair growth. Additionally, Minoxidil's vasodilatory properties may contribute to a cleansing effect on the pores, creating a favorable environment for hair follicles to thrive. This formulation harnesses the vasodilating power of Minoxidil to support individuals seeking to rejuvenate and maintain a healthy scalp.

Transferosomes are similar to ethosomes but also contain surfactants that enhance their flexibility and deformability, allowing them to pass through the skin's barrier more effectively, such as down hair follicles and acne pores via capillary action. The composition can include surfactants such as with Polyhexamethylene Biguanide (PHMB), benzalkonium chloride, betaine, other quaternary ammonias, or other surfactants.

Transferosomes are specialized lipid-based vesicles designed for enhanced transdermal drug delivery. These vesicles exhibit flexibility, allowing them to deform and squeeze through pores smaller than their diameters, making them particularly effective in penetrating the skin's lipid barrier. These surfactants contribute to the amphipathic nature of transferosomes, facilitating their interaction with both hydrophobic and hydrophilic components.

To enhance efficacy in applications targeting pores, hair follicles, acne, and hair loss, transferosomes are leveraged for their capillary action, and exhibit notable advantages. This capillary action facilitates the downward movement of the solution through hair follicles, ensuring effective penetration into pores and follicles. This innovative approach significantly improves the delivery of the formulation, providing precise and targeted solutions for conditions such as acne and hair loss. The integration of transferosomes not only optimizes therapeutic outcomes for general dermatological applications but also presents a novel and effective topical delivery system specifically tailored to address concerns related to acne and hair loss.

### Novel Formulations Incorporating Select Medications Into An Ethosome Mixture For Various Topically Addressable Ailments

The following are further exemplars of conditions to be treated by medications that can be encapsulated and carried through the fatty bilayers of the skin as ethosome mixtures, integrating novel ethosome-drug combinations. Capitalizing on their inherent penetrating characteristics, these formulations efficiently reach the skin, follicles, pores, and sweat glands. Notably, they showcase an amplified capacity to penetrate keratin and delve into deeper skin structures, leveraging the methodologies outlined in this context.

The compositions may be applied to the desired site for delivery of the active ingredients. Generally, the application is topical to the skin, but the compositions may also be used intraoperatively. Additionally, the compositions may be incorporated in predetermined therapeutically effective amounts into disposables such as wipes, gauze, patches, wraps, bandages, adhesive strips, sponge, cotton swab, sutures, medication pad, etc.

### Hidradenitis Suppurativa

Trimethoprim/Sulfamethoxazole (TMP/SMX or Bactrim)™ and Clindamycin can be utilized to formulate a treatment for Hidradenitis Suppurativa (HS). HS is a dermatological condition characterized by painful lumps deep within the skin, often forming in areas where skin contacts skin, such as the armpits, inner thighs, and groin. These lumps result from clogged hair and sweat glands. The formulations presented herein, namely incorporating Bactrim™ and Clindamycin as the active drug, address HS by facilitating the self-draining of abscesses, eliminating the need for surgical intervention. The unique composition penetrates the ducts in glands, providing a non-invasive solution for this condition.

In cases of HS where the pores or follicles become clogged, traditional treatments often involve surgery. The formulation discussed allows for drainage through the natural duct, avoiding repetitive incisions for draining an abscess and associated scarring. This embodiment offers an innovative approach, preventing the need for surgical procedures and ensuring effective relief for individuals suffering from HS.

A formulation of a preferred embodiment using HS (hidradenitis suppurativa) and acne antibiotic gel is as follows. Add 1 gram of crushed clindamycin pills to 3 grams of crushed triple sulfur antibiotic (5 pills). To this add 3-10 grams ethanol and mix until dissolved. To this add 180-300 ml of the heated mixture of isopropyl alcohol and 1% soy lecithin. Add 90-180 ml propylene glycol. Stir. Then shock with 180 ml hot distilled water. Then PHMB 1 grams or 5 ml of 20%. mix. To this add enough methyl and ethyl cellulose gel to make one liter.

This is used to penetrate pores and hair follicles for acne, Cystic Acne and cysts of a hydranitis supuritiva blocked cyst hair follicle inverted.

### Acne

Acne can be treated with a 0.01 to 0.1 g/L retinoic acid ethosome formulation and cleans out clogged pores. In a preferred embodiment 0.05 g/L is used. This innovative formulation acts to penetrate easily into and cleanse clogged pores effectively, providing a targeted approach to address acne-related concerns, like infections.

### Pain Relievers

A pain-relieving formula incorporating any one, or a combination of Benadryl®, Tramadol, Vicodin®, Ibuprofen, Valium®, or neuropathy Neurontin®, can be created, providing the user more precise control over dose and target area. In an alternative embodiment, a combination of Benadryl®, Tramadol, Vicodin®, Ibuprofen, Valium®, and with neuropathy Neurontin® can be incorporated as the active medications in the ethosomal formulation.

This distinctive formulation incorporates Benadryl® to address allergic reactions and enhance the overall pain-relieving properties of the formula. Tramadol and Vicodin®, both powerful analgesics, contribute to the formula's efficacy in managing moderate to severe pain. The inclusion of Ibuprofen provides anti-inflammatory benefits, addressing pain associated with inflammation. Valium®, a muscle relaxant and anxiolytic, complements the formula by alleviating muscle tension and anxiety often linked to chronic pain conditions. Neurontin®, specifically targeted at neuropathic pain, enhances the formula's ability to address nerve-related discomfort effectively.

### Otc Analgesics

Trolamine salicylate, or other OTC analgesics, akin to aspirin, is included for its anti-inflammatory and pain-relieving effects in topical analgesics. Isopropyl alcohol, a quick-evaporating solvent, induces a cooling sensation and aids in ingredient penetration. The ethosome base ensures effective absorption of active ingredients into the skin, enhancing the overall efficacy of the formulation. Peppermint, known for its cooling sensation and potential analgesic properties, offers a soothing effect, helping alleviate pain or discomfort. The result is a cooling and pain-relieving effect on the skin, providing a targeted and controlled solution for pain relief. The formulation may incorporate 15-20% glycerin, 15% peppermint, 5-10% trolamine salicylate, and isopropyl alcohol in an ethosome mixture as described above.

A formulation of a preferred embodiment using Pain OTC ethosome formulation analgesic spray includes 60-70% alcohol with 1% soy lecithin heated to about 86° F.). (30° C., 15-20% glycerine, 10% triethanolamine salicylate, 5% peppermint oil.

### Migraines

Rizatriptan serves as the active medication in formulating a topical treatment for migraines to be applied over the trigeminal nerve. This groundbreaking approach directly addresses migraines by specifically targeting the trigeminal nerve with the innovative application of rizatriptan. Notably, this formulation stands out as an advanced therapeutic option, aiming to effectively alleviate migraine headaches with precision while utilizing a lower dose than previously required.

In contrast to conventional oral treatments, this formulation offers a localized and non-invasive alternative for migraine relief. By minimizing systemic exposure, it reduces the risk of potential side effects associated with traditional oral medications. The formulation's efficacy is highlighted by its focused impact on the trigeminal nerve, a crucial factor in migraine pathophysiology.

The unique approach of this formulation positions it as an innovative and patient-centered solution for individuals seeking effective migraine management. The topical application of rizatriptan not only showcases the potential for enhanced efficacy but also emphasizes a reduced systemic impact in addressing the challenges posed by migraines.

### Eczema

A formulation incorporating methotrexate can be used for treatment of psoriasis and eczema. Methotrexate is a medication that is sometimes used in the treatment of psoriasis and eczema, although it is more commonly associated with psoriasis. Psoriasis and eczema are both chronic inflammatory skin conditions, and methotrexate can help manage symptoms in certain cases.

In the context of psoriasis, methotrexate is considered a systemic treatment, meaning it affects the entire body. It works by inhibiting the rapid growth of skin cells that occurs in psoriasis, helping to reduce inflammation and slow down the progression of the condition. Methotrexate is often prescribed when psoriasis is widespread or has not responded well to other treatments. While methotrexate is less commonly used for eczema, it may now be considered a viable treatment, as opposed to only for severe cases due to potential side effects associated with its systemic action.

It's important to note that the use of methotrexate for these skin conditions usually needs to be carefully monitored by healthcare professionals, as it can have side effects and may not be suitable for everyone. The use of methotrexate utilizing the methods and formulations presented herein allows topical applications of methotrexate without concern as to the levels in the user's blood or toxicity. Substantially smaller amounts are needed per application when utilizing the methods herein, as the medication penetrates through the skin and targets the affected area directly, rather than being present in large amounts in the bloodstream.

### Antifungal

This formulation integrates antifungal agents, including ciclopirox olamine, tolnaftate, terbinafine HCl, and clotrimazole, into an ethosome base tailored to target fungal concerns, particularly for nails. Ethosomes, acting as a lipid-based delivery system, enhance the permeation of these active ingredients through the nail and surrounding skin. This amplifies absorption and efficacy of the antifungal agents.

Ciclopirox olamine, effective against nail infections, tolnaftate with potent antifungal properties, terbinafine HCl combating a broad spectrum of fungi, and clotrimazole with activity against various fungi collectively provide a comprehensive approach. Natural ingredients like peppermint, eucalyptus globulus, and pine contribute not only a pleasant fragrance but also potential benefits. Peppermint offers cooling properties, while eucalyptus and pine are linked to antimicrobial and soothing effects. Mineral oil and/or petrolatum can also be included for their emollient properties, aiding in moisturizing and softening the nails and surrounding skin, promoting overall nail health and appearance. This carefully curated combination facilitates a stable mixture.

With a focus on enhanced penetration through ethosomes, this formulation strives to offer an effective solution for fungal nail infections. The emphasis on targeted delivery aims to improve the distribution of active ingredients in affected areas while minimizing systemic concentrations.

A formulation of a preferred embodiment using an antifungal formula with petrolatum is as follows. Antifungals including tolnaftate, clotrimazole, terbinafine or a combination is dissolved in ethanol. To this 1000 ml alcohol (isopropyl) is heated between 30 and 40° C. To this 30-90 ml of soy lecithin is added and mixed and dissolved. Approx. 480 ml is added to the dissolved antifungal. Then 360 ml of propylene glycol is added. Then 90 ml of PHMB. The mixture is placed in a 5 gallon container. To this 1440-1800 ml of petrolatum is added. Then, approximately half or two gallons of mineral oil is added. Then mix with an electric mixer. Then the other half or two gallons of mineral oil is added and mixed for a total of four gallons of mineral oil. This makes a liquid gel ethosome mixture liquid/gel topically applied to toenails or skin with a small brush.

### Suitable Medications for Use in Ethosomes

Below is a partial list of active drugs or additives with suggested dosing that can be used in the composition. Other suitable compositions would be known to those skilled in the art. It is understood that the proportions of ingredients may be adjusted to enhance different characteristics and such adjustments would be optimized by those skilled in the art. Typically one applies a small amount (such as 30 ml) over the affected area 1-2×per day or as similarly directed by their treating physician

| | Concentration/Dosing | | |
|---|---|---|---|
| Drug | per liter of carrier | | Purpose |
| Cyclosporine A (CA) | 100 mg-1 g | | psoriasis and eczema |
| Trimethoprim/ | 800 mg-1200 mg | | Hidradenitis Suppurativa |
| Sulfamethoxazole | 400 mg-1200 mg (900 mg) | | Acne |
| (Bactrim ™) & | | | |
| Clindamycin | | | |
| Retinoic Acid | .1-.01 | g | Acne, aging skin |
| Tramadol | 200 mg-1000 mg | | pain reliever |
| Vicodin ® | 5-100 | mg | pain reliever |
| Ibuprofen | 1200-2400 | mg | pain reliever |
| Valium ® | 60-120 | mg | pain reliever & muscle |
| | | | relaxant |
| Neurontin ® | 1-3 | mg | pain reliever & nerve |
| | | | relaxant |
| Rizatriptan | 60-120 | mg | migraines |
| Methotrexate | 15-20 | mg | psoriasis and eczema |

| | | |
|---|---|---|
| Trolamine Salicylate | 5-10% | pain reliever |
| Ciclopirox Olamine | 1-4% | antifungal |
| Tolnaftate | 1-4% | antifungal |
| Terbinafine HCl | 1-4% | antifungal |
| Clotrimazole | 1-4% | antifungal |
| Eucalyptus Globulus | 1-5% | antifungal |
| Finasteride | .3% | Hair loss formulation |
| Minoxidil | 2-5% | Hair loss formulation |

The innovative ethosome formulation may also be used with the active ingredients disclosed in Pat. Nos. 8,580,264 & 10,874,677 as well as those used in commercially available treatments such as other active ingredients used in Humera®, Skyrizi®, Stalara®, Actema®, Cosentyx® adaptable to those skilled in the art. Some preferred dosing ranges are given below.

| | | | |
|---|---|---|---|
| Humera ® (Adalimumab) | 40 | mg | Monoclonal antibodies |
| | | | TNF Blocker |
| Skyrizi ® (Risankizumab) | 150 | g | TNP Blocker |
| | | | Interleukin 23 inhibitor |
| Stelara ® (Ustekinumab) | 40-90 | mg | Monoclonal antibodies |
| | | | Interleukin 12 and 23 |
| Actemra ® (Tocilizumab) | 160-400 | mg | Monoclonal antibodies |
| | | | Immune suppression |
| | | | Interleukin -6 |
| Cosentyx ® | 150 | g | Interleukin -17A |

(Secukinumab)

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A transdermal drug delivery composition for use by an animal having skin comprising a stratum corneum layer and a dermis layer, said composition utilized to deliver an effective amount of a pharmacological agent through said stratum corneum layer of the skin comprising:
a) a plurality of ethosomes with a maximum length range of 20-400 nm, wherein an ethosome comprises
(i) a volume of alcohol and
(ii) a phospholipid fully dissolved in said volume of alcohol
b) a pharmaceutically active penetrating solution comprising
i) a volume of ethanol
ii) said pharmacological agent fully dissolved in said volume of ethanol
wherein the pharmaceutically penetrating active solution is encapsulated in a plurality of said ethosomes and wherein the ethosomes and active agent can penetrate the stratum corneum and dermis layer of skin, wherein the ethosomes are non-polar and fat soluble, wherein the alcohol is isopropyl alcohol or ethanol.

2. The composition according to claim 1 wherein the phospholipid is dissolved into the isopropyl alcohol to make a phospholipid solution and wherein the phospholipid is soy lecithin.

3. The composition according to claim 2 wherein the phospholipid solution further comprises propylene glycol to enhance stability and provide a vehicle for drug distribution.

4. The composition according to claim 1 wherein the pharmaceutical agent is fully dissolved into ethanol.

5. The composition according to claim 1 further comprises at least one or more of the ingredients selected from the group consisting of glycerin, PHMB (Polyhexamethylene Biguanide), caprylic acid and glycol, and cellulose gel.

6. A method for preparing a lipid-based vesicle formulation for enhanced skin penetration, comprising the steps of:
a. preparing a lipid-alcohol solution, by,
i. warming a predetermined volume of alcohol,
ii. adding a predetermined amount of lipid to result in a 1-4% solution,
iii. mixing said volume of alcohol and lipid,
b. solubilizing a predetermined amount of medicament by dissolving the medicament in alcohol,
c. mixing a predetermined amount of the lipid-alcohol solution with said solubilized medicament resulting in a solubilized lipid-alcohol-medicament solution,
d. mixing in a predetermined amount of polyhydric alcohol,
e. mixing in a predetermined amount of water, causing the solubilized lipid-alcohol-medicament solution to undergo lipid vesicle formation and encapsulation of the medicament within said lipid vesicles, and
f. preparing a gel suspension for retention of said medicament filled lipid vesicles.

7. The method of claim 6, wherein the ethosome mixture is put into an ultrasound prior to addition of hot water and shocking the mixture.

8. The method of claim 6 further comprises at least one or more of the ingredients selected from the group consisting of pure cyclosporine A, trimethoprim sulfamethoxazole, clindamycin, retinoic acid, rizatriptan, methotrexate, minoxidil, finasteride, an antifungal, adalimumab, risankizumab, ustekinumab, tocilizumab, secukinumab and an analgesic.

9. The method of claim 8, wherein the antifungal comprises at least one or more of the ingredients selected from the group consisting of ciclopirox olamine, tolnaftate, terbinafine HCl, clotrimazole and peppermint, eucalyptus globulus and pine with mineral oil.

10. The method of claim 8 wherein the analgesic comprises at least one or more of the ingredients selected from the group consisting of comprises glycerin, peppermint, and trolamine salicylate.

11. The method of claim 10, wherein the pain reliever is at least one or more of the ingredients selected from the group consisting of diphenhydramine, tramadol, hydrocodone, ibuprofen, diazepam, and gabapentin.
